(19) United States
(12) Patent Application Publication
(10) Pub. No.: US 2007/0264343 A1
(43) Pub. Date: Nov. 15, 2007

(54) METHODS FOR MAKING AND USING PARTICULATE PHARMACEUTICAL FORMULATIONS FOR SUSTAINED RELEASE

(75) Inventors: Howard Bernstein, Cambridge, MA (US); Donald E. Chickering III, Framingham, MA (US); Eric K. Huang, Cambridge, MA (US); Sridhar Narasimhan, Elgin, IL (US); Shaina Reese, Winchester, MA (US); Julie A. Straub, Winchester, MA (US)

Correspondence Address: SUTHERLAND ASBILL & BRENNAN LLP, 999 PEACHTREE STREET, N.E., ATLANTA, GA, 30309, US

(73) Assignee: ACUSPHERE, INC., Watertown, MA (US)

(21) Appl. No.: 11/829,629

(22) Filed: Jul. 27, 2007

Related U.S. Application Data
(62) Division of application No. 10/950,856, filed on Sep. 27, 2004.
(60) Provisional application No. 60/507,384, filed on Sep. 30, 2003.

(51) Int. Cl. A61K 9/14; A61K 31/56; A61K 31/7088; A61K 38/00; A61K 9/10

(52) U.S. Cl. 424/486; 424/484; 514/169; 514/2; 514/44

(57) ABSTRACT

Pharmaceutical formulations and methods are provided for the sustained delivery of a pharmaceutical agent to a patient by injection. The injectable formulation includes porous microparticles which comprise a pharmaceutical agent and a matrix material, wherein upon injection of the formulation a therapeutically or prophylactically effective amount of the pharmaceutical agent is released from the microparticles for at least 24 hours. A method for making the injectable, sustained release pharmaceutical formulation may include dissolving a hydrophobic matrix material in a volatile solvent to form a first solution; adding a pharmaceutical agent to the first solution to form an emulsion, suspension, or second solution; and removing the volatile solvent from the emulsion, suspension, or second solution to yield porous microparticles which comprise the pharmaceutical agent dispersed, entrapped or encapsulated within the structure of the hydrophobic matrix material.

### CROSS-REFERENCE TO RELATED APPLICATIONS

This is a divisional of U.S. application Ser. No. 10/950,856, filed Sep. 27, 2004, now pending. Priority is claimed to U.S. Provisional Application No. 60/507,384, filed Sep. 30, 2003. These applications are incorporated herein by reference.

### BACKGROUND OF THE INVENTION

This invention is generally in the field of pharmaceutical formulations, and more particularly to microparticulate formulations for sustained release of pharmaceutical agents.

Current delivery systems are not ideal, often delivering inaccurate doses, requiring frequent dosing which discourages patient compliance. In addition, frequent dosing of immediate release formulations leads to pharmaceutical agent levels that peak and trough, causing undesirable toxicity or inadequate efficacy.

To deliver sustained release microparticulate pharmaceutical agents, compounds must be precisely formulated to ensure that they deliver the correct amount of pharmaceutical agent over the appropriate amount of time. This requires control of key factors such as geometric particle size and density and compatibility with select delivery devices and pharmaceutically acceptable carriers.

Conventional efforts towards sustained release particles have focused on the use of complexing agents, such as complexing a polycationic agent with a therapeutic agent. This approach, however, requires the therapeutic agent to be able to form a complex with the polycationic agent, which limits the therapeutic agents to anionic compounds. This approach also requires the polycation complexing agent to be non-toxic. This approach also has limited ability to control the release rate of the compound from the complex, as the release rate is essentially dependent upon the binding strength of the compound to the polycation.

Others have focused on designing formulations to control release via encapsulating a pharmaceutical agent within a polymer matrix, without porosity control. This approach has the disadvantage in that there are typically at least three release phases: an immediate release burst phase, a lag phase during which little drug is released, and a sustained phase in which the drug is release via a matrix degradation process. Oftentimes, the lag phase is undesirable because therapeutically effective amounts of the drug are not released during this phase.

It would be desirable to provide a sustained release, microparticle formulation of pharmaceutical agents, for local or systemic delivery by injection, or by oral or topical administration. It also would be desirable to provide a microparticle formulation of pharmaceutical agent enabling less frequent dosing.

### SUMMARY OF THE INVENTION

Sustained release pharmaceutical formulations are provided for delivery by injection, or by oral or topical administration. The formulations include porous microparticles which comprise a pharmaceutical agent and a matrix material.

In one aspect, a sustained release pharmaceutical formulation is provided for delivery to a patient by injection comprising porous microparticles that include a pharmaceutical agent and a matrix material, wherein a therapeutically or prophylactically effective amount of the pharmaceutical agent is released from the microparticles for at least 24 hours following injection of the formulation. In one embodiment, the porous microparticles have a volume average diameter between about 1 μm and 150 μm, e.g., between about 5 μm and 25 μm. In one embodiment, the porous microparticles have an average porosity between about 5 and 90% by volume. In one embodiment, the porous microparticles further comprise one or more surfactants, such as a phospholipid.

In one embodiment, the microparticles are dispersed in a pharmaceutically acceptable vehicle for injection. The vehicle can be aqueous or non-aqueous.

The formulation can include a wide range of pharmaceutical agents. For instance, the pharmaceutical agent can be a peptide, a protein, or an oligonucleotide. In various embodiments, the pharmaceutical agent comprises a steroid, an antipsychotic agent, an antineoplastic, or an antiemetic.

In various embodiments, the matrix material comprises a biocompatible synthetic polymer, a lipid, a hydrophobic molecule, or a combination thereof. For example, the synthetic polymer can comprise, for example, a polymer selected from the group consisting of poly(hydroxy acids) such as poly(lactic acid), poly(glycolic acid), and poly(lactic acid co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers, derivatives and blends thereof. In a preferred embodiment, the synthetic polymer comprises a poly(lactic acid), a poly(glycolic acid), a poly(lactic-co-glycolic acid), or a poly(lactide-co-glycolide).

In one embodiment, the formulation further comprises one or more other pharmaceutical agents. In one embodiment, the formulation further comprises additional microparticles blended with the porous microparticles. The additional microparticles can comprise one or more other pharmaceutical agents.

In another aspect, a method is provided for delivering a pharmaceutical agent to a patient comprising administering to the patient by injection a sustained release pharmaceutical formulation which comprises porous microparticles which comprise a pharmaceutical agent and a matrix material, wherein upon injection of the formulation a therapeutically or prophylactically effective amount of the pharmaceutical agent is released from the microparticles into the patient for at least 24 hours. Exemplary routes/sites of injection include intravenous, intraarterial, intracardiac, intrathecal, intraosseous, intraarticular, intrasynovial, intracutaneous, subcutaneous, intramuscular, and intradermal administration, as well as intracranial, intralesional, or intratumoral administration.

In one embodiment, a majority of the pharmaceutical agent is released from the microparticles by 14 days, 28 days, or 6 months following injection. In one embodiment, a majority of the pharmaceutical agent is release no earlier than about 24 hours and no later than about 28 days following injection. In a preferred embodiment, the formulation provides local or plasma concentrations which do not fluctuate by more than a factor of four over the period of sustained release. In various embodiments, a therapeutically or prophylactically effective amount of the pharmaceutical agent is released from the microparticles for at least 7 days following injection, for at least 14 days following injection, or for at least 28 days following injection.

In yet another aspect, methods are provided for making an injectable formulation for administration and sustained release of pharmaceutical agent. In a preferred embodiment, the method comprises the steps of: dissolving a matrix material in a volatile solvent to form a solution; adding a pharmaceutical agent to the solution to form an emulsion, suspension, or second solution; and removing the volatile solvent from the emulsion, suspension, or second solution to yield porous microparticles which comprise the pharmaceutical agent and the matrix material, wherein upon injection of the formulation a therapeutically or prophylactically effective amount of the pharmaceutical agent is released from the microparticles for at least 24 hours. In one embodiment, the method further comprises combining one or more surfactants with the solution. In one embodiment, the method further comprises combining the microparticles with a pharmaceutically acceptable vehicle for injection.

In another preferred embodiment, the method for making an injectable formulation for administration and sustained release of pharmaceutical agent comprises: dissolving a matrix material in a volatile solvent to form a solution; adding a pharmaceutical agent to the solution; combining at least one pore forming agent with the pharmaceutical agent in the solution to form an emulsion, suspension, or second solution; and removing the volatile solvent and the pore forming agent from the emulsion, suspension, or second solution to yield porous microparticles which comprise the pharmaceutical agent and the matrix material, wherein upon injection of the formulation a therapeutically or prophylactically effective amount of the pharmaceutical agent is released from the microparticles for at least 24 hours. The pore forming agent (e.g., a volatile salt) can be in the form of an aqueous solution when combined with the solution comprising matrix material. In one embodiment, the step of removing the volatile solvent and pore forming agent from the emulsion, suspension, or second solution is conducted using a process selected from spray drying, evaporation, fluid bed drying, lyophilization, vacuum drying, or a combination thereof.

In one aspect, a kit of parts is provided which comprises: a dry powder pharmaceutical formulation comprising porous microparticles which comprise a pharmaceutical agent and a matrix material; and a pharmaceutically acceptable vehicle for injection, wherein upon mixing of the dry powder pharmaceutical formulation into the pharmaceutically acceptable vehicle to form an injectable formulation and then injecting of the injectable formulation, a therapeutically or prophylactically effective amount of the pharmaceutical agent is released from the microparticles for at least 24 hours.

In yet another aspect, a sustained release pharmaceutical formulation for delivery to a patient by oral administration is provided. The formulation includes porous microparticles which comprise a pharmaceutical agent and a matrix material, wherein a therapeutically or prophylactically effective amount of the pharmaceutical agent is released from the microparticles for at least 2 hours, at least 4 hours, at least 8 hours, at least 16 hours, or at least 24 hours, following oral administration of the formulation. In one embodiment, the matrix material is selected from biocompatible synthetic polymers, lipids, hydrophobic compounds, or combinations thereof. In one embodiment, the microparticles are combined with one or more pharmaceutically acceptable additives for oral administration. Methods are provided for delivering a pharmaceutical agent to a patient comprising orally administering to a patient a sustained release pharmaceutical formulation that includes porous microparticles which comprise a pharmaceutical agent and a matrix material, wherein a therapeutically or prophylactically effective amount of the pharmaceutical agent is released from the microparticles into the patient for at least 2 hours following oral administration.

In still another aspect, a sustained release pharmaceutical formulation for delivery to a patient by topical administration is provided. The formulation includes porous microparticles which comprise a pharmaceutical agent and a matrix material, wherein a therapeutically or prophylactically effective amount of the pharmaceutical agent is released from the microparticles for at least 2 hours, for at least 12 hours, for at least 24 hours, for at least 2 days, or for at least 7 days, following topical administration to the patient. In one embodiment, the matrix material is selected from biocompatible synthetic polymers, lipids, hydrophobic materials, or combinations thereof. In one embodiment, the microparticles are combined with one or more pharmaceutically acceptable additives for topical administration. Methods are provided for delivering a pharmaceutical agent to a patient comprising topically administering to the patient a sustained release pharmaceutical formulation which comprises porous microparticles which comprise a pharmaceutical agent and a matrix material, wherein a therapeutically or prophylactically effective amount of the pharmaceutical agent is released from the microparticles into the patient for at least 2 hours following topical administration.

### BRIEF DESCRIPTION OF THE DRAWINGS

FIG. 1 is a graph of percent in vitro release of budesonide after 5.5 hours versus percent porosity of the microparticles.

FIG. 2 is a graph of percent in vitro release of fluticasone propionate after 5.5 hours versus percent porosity of the microparticles.

FIG. 3 is a graph of percent in vitro release of fluticasone propionate after 24 hours versus percent porosity of the microparticles.

### DETAILED DESCRIPTION OF THE INVENTION

An injectable, oral, or topical sustained release delivery system for pharmaceutical agents has been developed. The delivery system is a formulation comprising porous microparticles, where porosity, particle geometric diameter and composition are selected and used to control the rate of release of pharmaceutical agent from the microparticles following injection, oral administration or topical administration. In particular, it has been discovered that the composition of the microparticles (e.g., the matrix material, surfactant) can be selected to provide delayed release (and avoid the burst effect associated with immediate release formulations), and the porosity of the microparticles can be selected to provide additional control of the rate of release of the pharmaceutical agent and to provide continuous release of the majority of the pharmaceutical agent, avoiding a lag phase after administration. Although the composition of the microparticles can be selected to slow the release of the pharmaceutical agent, selection of the composition alone may not ensure that an appropriate amount of the pharmaceutical agent is released continuously over the desired duration following administration. For a given composition of the microparticles, the porosity can be selected to ensure that a therapeutically or prophylactically effect amount of the pharmaceutical agent continues to be released continuously for at least 24 hours following injection, or at least 2 hours following oral or topical administration.

Advantageously, the porous microparticles can provide sustained local delivery of pharmaceutical agent and/or sustained plasma levels without the need to complex the pharmaceutical agent molecule with another molecule. In addition, the sustained delivery formulations advantageously can moderate the pharmaceutical agent peaks and troughs associated with immediate release pharmaceutical agents, which can cause added toxicity or reduced efficacy.

Advantageously, the method and formulation can provide local or plasma concentrations at approximately constant values. For example, they may not fluctuate by more than a factor of four over the period of sustained release.

As used herein, the terms “comprise,” “comprising,” “include,” and “including” are intended to be open, non-limiting terms, unless the contrary is expressly indicated.

The Sustained Release Formulations

The sustained release pharmaceutical formulations for parenteral administration include porous microparticles that comprise a pharmaceutical agent and a matrix material. The microparticle's composition, geometric diameter, and porosity provide that upon administration of the formulation a therapeutically or prophylactically effective amount of the pharmaceutical agent is released in a sustained manner from the microparticles in the body over a duration that extends up to at least about 24 hours after injection or at least 2 hours after oral administration or topical administration.

In one embodiment, a majority of the pharmaceutical agent is released by about 14 days after administration via injection. In another embodiment, a majority of the pharmaceutical agent is released by about 28 days after administration via injection.

In one embodiment, a majority of the pharmaceutical agent is released by about 24 hours after oral administration or topical administration. In another embodiment, a majority of the pharmaceutical agent is released by about 7 days after topical administration.

As a measure of sustained release, the mean absorption time following administration (MAT_{adm}) for the drug can be used. The MAT_{adm} is the average time it takes for a drug molecule to be absorbed into the bloodstream following administration and can be calculated from the pharmaceutical agent plasma profile following administration as follows:
MAT_{adm}=(AUMC_{adm∞}/AUC_{adm∞})−MRTᵢᵥ (EQ. 1)
where AUMC_{adm∞} is area under the first moment curve (product of time and plasma concentration) from time zero to infinity following administration, AUC_{adm∞} is the area under the plasma concentration curve from time zero to infinity following administration, and MRTᵢᵥ is the mean residence time for the pharmaceutical agent of interest following intravenous administration. The MRTᵢᵥ can be determined as follows:
MRTᵢᵥ=(AUMC_{iv∞}/AUC_{iv∞}) (EQ. 2)
where AUMC_{iv∞} is area under the first moment curve (product of time and plasma concentration) from time zero to infinity following intravenous administration, and AUC_{iv∞} is the area under the plasma concentration curve from time zero to infinity following intravenous administration.

For example, the porous microparticles can provide a pharmaceutical agent mean absorption time following administration greater than the pharmaceutical agent mean absorption time following administration when not delivered in microparticle form. The desired MAT_{adm} will depend on the drug molecule to be administered, and it is helpful to consider the increase in MAT_{adm} obtained using the present microparticle formulations compared to the drug molecule when not delivered as microparticles. In preferred embodiments, a drug administered in microparticles of the present compositions and methods will provide an increase in MAT_{adm} of at least between about 25 and 50% as compared to the drug administered not in the present microparticles.

The sustained release formulations are achieved by controlling microparticle composition, microparticle geometric size, and microparticle porosity. Porosity (ε) is the ratio of the volume of voids contained in the microparticles (Vᵥ) to the total volume of the microparticles (Vₜ):
ε=Vᵥ/Vₜ (EQ. 3)
This relationship can be expressed in terms of the envelope density (ρₑ) of the microparticles and the absolute density (ρₐ) of the microparticles:
ε=1−ρₑ/ρₐ (EQ. 4)

The absolute density is a measurement of the density of the solid material present in the microparticles, and is equal to the mass of the microparticles (which is assumed to equal the mass of solid material, as the mass of voids is assumed to be negligible) divided by the volume of the solid material (i.e., excludes the volume of voids contained in the microparticles and the volume between the microparticles). Absolute density can be measured using techniques such as helium pycnometry. The envelope density is equal to the mass of the microparticles divided by the volume occupied by the microparticles (i.e., equals the sum of the volume of the solid material and the volume of voids contained in the microparticles and excludes the volume between the microparticles). Envelope density can be measured using techniques such as mercury porosimetry or using a GeoPye™ instrument (Micromeritics, Norcross, Ga.). The envelope density can be estimated from the tap density of the microparticles. The tap density is a measurement of the packing density and is equal to the mass of microparticles divided by the sum of the volume of solid material in the microparticles, the volume of voids within the microparticles, and the volume between the packed microparticles of the material. Tap density (pt) can be measured using a GeoPye™ instrument or techniques such as those described in the British Pharmacopoeia and ASTM standard test methods for tap density. It is known in the art that the envelope density can be estimated from the tap density for essentially spherical microparticles by accounting for the volume between the microparticles:
ρₑ=ρₜ/0.794 (EQ.5)
The porosity can be expressed as follows:
ε=1−ρₜ/(0.794*ρₐ) (EQ.6)

For a given microparticle composition (pharmaceutical agent and matrix material) and structure (microparticle porosity and thus density) an iterative process can be used to define the duration over which the microparticles release the pharmaceutical agent: (1) the matrix material, the pharmaceutical agent content, and the microparticle geometric size are selected to determine the time and amount of initial pharmaceutical agent release; (2) the porosity of the microparticles is selected to adjust the amount of initial pharmaceutical agent release, and to ensure that significant release of the pharmaceutical agent occurs beyond the initial release; and then (3) the geometric particle size and the porosity are adjusted to facilitate administration by the selected route and to exhibit any necessary or desirable characteristics at the injection site, for example, to avoid or delay any physiological clearance mechanisms that would remove the microparticles from the injection site prior to their releasing substantially all of the pharmaceutical agent contained therein. As used herein, the term “initial release” refers to the amount of pharmaceutical agent released shortly after the microparticles become wetted. The initial release upon wetting of the microparticles results from pharmaceutical agent which is not fully encapsulated and/or pharmaceutical agent which is located close to the exterior surface of the microparticle. The amount of pharmaceutical agent released in the first 10 minutes is used as a measure of the initial release.

As used herein, the terms “diameter” or “d” in reference to particles refers to the number average particle size, unless otherwise specified. An example of an equation that can be used to describe the number average particle size is shown below:

$$d = \frac{{\sum }_{i = 1}^{p} {n}_{i} {d}_{i}}{{\sum }_{i = 1}^{p} {n}_{i}} \qquad ( EQ . 7 )$$

where n number of particles of a given diameter (d).

As used herein, the terms “geometric size,” “geometric diameter,” “volume average size,” “volume average diameter” or “d_{g}” refers to the volume weighted diameter average. An example of equations that can be used to describe the volume average diameter is shown below:

$${d}_{g} = {[ \frac{{\sum }_{i = 1}^{p} {n}_{i} {d}_{i}^{3}}{{\sum }_{i = 1}^{p} {n}_{i}} ]}^{1 / 3} \qquad ( EQ . 8 )$$

where n=number of particles of a given diameter (d).

As used herein, the term “volume median” refers to the median diameter value of the volume-weighted distribution. The median is the diameter for which 50% of the total are smaller and 50% are larger, and corresponds to a cumulative fraction of 50%.

Geometric particle size analysis can be performed on a Coulter counter, by light scattering, by light microscopy, scanning electron microscopy, or transmittance electron microscopy, as known in the art.

The Porous Microparticles

The porous microparticles comprise a matrix material and a pharmaceutical agent. As used herein, the term “matrix” refers to a structure including one or more materials in which the pharmaceutical agent is dispersed, entrapped, or encapsulated. The matrix is in the form of porous microparticles. Optionally, the porous microparticles further include one or more surfactants.

As used herein, the term “microparticle” includes microspheres and microcapsules, as well as microparticles, unless otherwise specified. Microparticles may or may not be spherical in shape. Microcapsules are defined as microparticles having an outer shell surrounding a core containing another material, for example, the pharmaceutical agent. Microspheres comprising pharmaceutical agent and matrix can be porous having a honeycombed structure or a single internal void. Either type of microparticle may also have pores on the surface of the microparticle.

As used herein, microparticles are particles having a size of 0.5 to 1000 microns. In one embodiment, the microparticles have a volume average diameter between 1 and 150 μm (e.g., between 5 and 25 μm, between 10 and 25 μm, etc.). Different injection sites and administration routes typically indicate the desired size range within this broad range. In one embodiment, the volume average diameter is selected to avoid and minimize effects of the body's natural clearance mechanisms (e.g., phagocytosis by macrophages). Generally, larger particles are phagocytosed at a slower rate.

In one embodiment, the microparticles have an average porosity between about 5 and 90%. The porosity of the microparticles is selected so that the majority of the pharmaceutical agent is released within the desired duration of sustained release. In specific embodiments, the average porosity can be between about 25 and about 75%, between about 35 and about 65%, or between about 40 and about 60%.

Matrix Material

The matrix material is a material that functions to slow down release of the pharmaceutical agent from the microparticle. It can be formed of non-biodegradable or biodegradable materials, although biodegradable materials are often preferred.

The matrix material can be crystalline, semi-crystalline, or amorphous. The matrix material may be a polymer, a lipid, a salt, a hydrophobic small molecule, or a combination thereof.

The pharmaceutical agent can be present in the porous microparticle in an amount that is greater than or less than the amount of matrix material that is present in the porous microparticle, depending upon the particular formulation needs.

The matrix material comprises at least 5% w/w of the microparticle. The content of matrix material in the microparticles can be between 5 and about 95 wt %. In typical embodiments, the matrix material is present in an amount between about 50 and 90 wt %.

Representative synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polyvinyl acetate phthalate, polystyrene, polyurethanes and copolymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, hydroxypropyl methylcellulose phthalate, cellulose acetate trimellitate, carboxymethy ethylcellulose, hydroxypropylmethylcellulose acetate succinate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide co-caprolactone), copolymers, derivatives and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid (including poly(lactide-co-glycolide)), and copolymers with PEG, polyanhydrides, poly(ortho)esters, poly(butyric acid), poly(valeric acid), poly(lactide-co-caprolactone), blends and copolymers thereof.

Examples of preferred natural polymers include proteins such as albumin, fibrinogen, gelatin, and prolamines, for example, zein, and polysaccharides such as alginate, cellulose and polyhydroxyalkanoates, for example, polyhydroxybutyrate.

Representative lipids include the following classes of molecules: fatty acids and derivatives, mono-, di- and triglycerides, phospholipids, sphingolipids, cholesterol and steroid derivatives, terpenes, and vitamins. Fatty acids and derivatives thereof may include saturated and unsaturated fatty acids, odd and even number fatty acids, cis and trans isomers, and fatty acid derivatives including alcohols, esters, anhydrides, hydroxy fatty acids and prostaglandins. Saturated and unsaturated fatty acids that may be used include molecules that have between 12 carbon atoms and 22 carbon atoms in either linear or branched form. Examples of saturated fatty acids that may be used include lauric, myristic, palmitic, and stearic acids. Examples of unsaturated fatty acids that may be used include lauric, physeteric, myristoleic, palmitoleic, petroselinic, and oleic acids. Examples of branched fatty acids that may be used include isolauric, isomyristic, isopalmitic, and isostearic acids and isoprenoids. Fatty acid derivatives include 12-(((7′-diethylaminocoumarin-3 yl)carbonyl)methylamino)-octadecanoic acid; N-[12-(((7′diethylaminocoumarin-3-yl)carbonyl)methyl-amino)octadecanoyl]-2-aminopalmitic acid, N succinyl-dioleoylphosphatidylethanol amine and palmitoyl-homocysteine; and/or combinations thereof. Mono, di- and triglycerides or derivatives thereof that may be used include molecules that have fatty acids or mixtures of fatty acids between 6 and 24 carbon atoms, digalactosyldiglyceride, 1,2-dioleoyl-sn-glycerol; 1,2-dipalmitoyl-sn-3 succinylglycerol; and 1,3-dipalmitoyl-2-succinylglycerol.

In one preferred embodiment, the matrix material comprises a phospholipid or combinations of phospholipids. Phospholipids that may be used include phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phosphatidylcholines include such as dioleoylphosphatidylcholine, dimyristoylphosphatidylcholine (DMPC), dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) may also be used. Examples of phosphatidylethanolamines include dicaprylphosphatidylethanolamine, dioctanoylphosphatidylethanolamine, dilauroylphosphatidylethanolamine, dimyristoylphosphatidylethanolamine (DMPE), dipalmitoylphosphatidylethanolamine (DPPE), dipalmitoleoylphosphatidylethanolamine, distearoylphosphatidylethanolamine (DSPE), dioleoylphosphatidylethanolamine, and dilineoylphosphatidylethanolamine. Examples of phosphatidylglycerols include dicaprylphosphatidylglycerol, dioctanoylphosphatidylglycerol, dilauroylphosphatidylglycerol, dimyristoylphosphatidylglycerol (DMPG), dipalmitoylphosphatidylglycerol (DPPG), dipalmitoleoylphosphatidylglycerol, distearoylphosphatidylglycerol (DSPG), dioleoylphosphatidylglycerol, and dilineoylphosphatidylglycerol. Preferred phospholipids include DMPC, DPPC, DAPC, DSPC, DTPC, DBPC, DMPG, DPPG, DSPG, DMPE, DPPE, and DSPE.

Additional examples of phospholipids include modified phospholipids for example phospholipids having their head group modified, e.g., alkylated or polyethylene glycol (PEG)-modified, hydrogenated phospholipids, phospholipids with multifarious head groups (phosphatidylmethanol, phosphatidylethanol, phosphatidylpropanol, phosphatidylbutanol, etc.), dibromo phosphatidylcholines, mono and diphytanoly phosphatides, mono and diacetylenic phosphatides, and PEG phosphatides.

Sphingolipids that may be used include ceramides, sphingomyelins, cerebrosides, gangliosides, sulfatides and lysosulfatides. Examples of sphinglolipids include the gangliosides GM1 and GM2.

Steroids which may be used include cholesterol, cholesterol sulfate, cholesterol hemisuccinate, 6-(5-cholesterol 3β-yloxy)hexyl-6-amino-6-deoxy-1-thio-α-D-galactopyranoside, 6-(5-cholesten-3 β-yloxy)hexyl-6-amino-6-deoxyl-1-thio-α-D mannopyranoside and cholesteryl(4′-trimethyl 35 ammonio)butanoate.

Additional lipid compounds that may be used include tocopherol and derivatives, and oils and derivatized oils such as stearlyamine.

Other suitable hydrophobic compounds include amino acids such as tryptophane, tyrosine, isoleucine, leucine, and valine, aromatic compounds such as an alkyl paraben, for example, methyl paraben, tyloxapol, and benzoic acid.

The matrix may comprise pharmaceutically acceptable small molecules such as carbohydrates (including mono and disaccharides, sugar alcohols and derivatives of carbohydrates such as esters), and amino acids, their salts and their derivatives such as esters and amides.

A variety of cationic lipids such as DOTMA, N-[1-(2,3-dioleoyloxy)propyl-N,N,N-trimethylammonium chloride; DOTAP, 1,2-dioleoyloxy-3-(trimethylammonio)propane; and DOTB, 1,2-dioleoyl-3-(4′-trimethyl-ammonio) butanoyl-sn glycerol may be used.

Inorganic materials can be included in the microparticles. Salts of metals (inorganic salts), such as calcium chloride or sodium chloride may be present in the particle or used in the production of the particles. Metal ions such calcium, magnesium, aluminum, zinc, sodium, potassium, lithium and iron may be used as the counterion for salts with organic acids such as citric acid and/or lipids including phospholipids. Examples of salts of organic acids include sodium citrate, sodium ascorbate, magnesium gluconate, and sodium gluconate. A variety of metal ions may be used in such complexes, including lanthanides, transition metals, alkaline earth metals, and mixtures of metal ions. Salts of organic bases may be included such as tromethamine hydrochloride.

In one embodiment, the microparticles may include one or more carboxylic acid as the free acid or the salt form. The salt can be a divalent salt. The carboxylate moiety can be a hydrophilic carboxylic acid or salt thereof. Suitable carboxylic acids include hydroxydicarboxylic acids, hydroxytricarboxylic acids and the like. Citric acid and citrate are preferred. Suitable counterions for salts include sodium and alkaline earth metals such as calcium. Such salts can be formed during the preparation of the particles, from the combination of one type of salt such as calcium chloride and carboxylic acid as the free acid or an alternative salt form such as the sodium salt.

In one embodiment, the porous microparticles further includes one or more surfactants. As used herein, a “surfactant” is a compound that is hydrophobic or amphiphilic (i.e., including both a hydrophilic and a hydrophobic component or region). Surfactants can be used to facilitate microparticle formation, to modify the surface properties of the microparticles and alter the way in which the microparticles are dispersed or suspended, to alter the properties of the matrix material (e.g. to increase or decrease the hydrophobicity of the matrix), or to perform a combination of functions thereof. It is to be distinguished from similar or identical materials forming the “matrix material.” The content of surfactant in the porous microparticles generally is less than about 10% by weight of the microparticles.

In one embodiment, the surfactant comprises a lipid. Lipids that may be used include the following classes of lipids: fatty acids and derivatives, mono-, di- and triglycerides, phospholipids, sphingolipids, cholesterol and steroid derivatives, terpenes, prostaglandins and vitamins. Fatty acids and derivatives thereof may include saturated and unsaturated fatty acids, odd and even number fatty acids, cis and trans isomers, and fatty acid derivatives including alcohols, esters, anhydrides, hydroxy fatty acids, and salts of fatty acids. Saturated and unsaturated fatty acids that may be used include molecules that have between 12 carbon atoms and 22 carbon atoms in either linear or branched form. Examples of saturated fatty acids that may be used include lauric, myristic, palmitic, and stearic acids. Examples of unsaturated fatty acids that may be used include lauric, physeteric, myristoleic, palmitoleic, petroselinic, and oleic acids. Examples of branched fatty acids that may be used include isolauric, isomyristic, isopalmitic, and isostearic acids and isoprenoids. Fatty acid derivatives include 12-(((7′-diethylaminocoumarin-3 yl)carbonyl)methylamino)-octadecanoic acid; N-[12-(((7′diethylaminocoumarin-3-yl)carbonyl)methyl-amino)octadecanoyl]-2-aminopalmitic acid, N succinyl-dioleoylphosphatidylethanol amine and palmitoyl-homocysteine; and/or combinations thereof. Mono, di- and triglycerides or derivatives thereof that may be used include molecules that have fatty acids or mixtures of fatty acids between 6 and 24 carbon atoms, digalactosyldiglyceride, 1,2-dioleoyl-sn-glycerol; 1,2-dipalmitoyl-sn-3 succinylglycerol; and 1,3-dipalmitoyl-2-succinylglycerol.

In one preferred embodiment, the surfactant comprises a phospholipid. Phospholipids that may be used include phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phosphatidylcholines include such as dioleoylphosphatidylcholine, dimyristoylphosphatidylcholine (DMPC), dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) may also be used. Examples of phosphatidylethanolamines include dicaprylphosphatidylethanolamine, dioctanoylphosphatidylethanolamine, dilauroylphosphatidylethanolamine, dimyristoylphosphatidylethanolamine (DMPE), to dipalmitoylphosphatidylethanolamine (DPPE), dipalmitoleoylphosphatidylethanolamine, distearoylphosphatidylethanolamine (DSPE), dioleoylphosphatidylethanolamine, and dilineoylphosphatidylethanolamine. Examples of phosphatidylglycerols include dicaprylphosphatidylglycerol, dioetanoylphosphatidylglycerol, dilauroylphosphatidylglycerol, dimyristoylphosphatidylglycerol (DMPG), dipalmitoylphosphatidylglycerol (DPPG), dipalmitoleoylphosphatidylglycerol, distearoylphosphatidylglycerol (DSPG), dioleoylphosphatidylglycerol, and dilineoylhosphatidylglycerol. Preferred phospholipids include DMPC, DPPC, DAPC, DSPC, DTPC, DBPC, DLPC, DMPG, DPPG, DSPG, DMPE, DPPE, and DSPE, and most preferably DPPC, DAPC and DSPC.

Sphingolipids that may be used include ceramides, sphingomyelins, cerebrosides, gangliosides, sulfatides and lysosulfatides. Examples of sphinglolipids include the gangliosides GM1 and GM2.

Steroids which may be used include cholesterol, cholesterol sulfate, cholesterol hemisuccinate, 6-(5-cholesterol 3β-yloxy)hexyl-6-amino-6-deoxy-1-thio-α-D-galactopyranoside, 6(5-cholesten-3β-yloxy)hexyl-6-amino-6-deoxyl-1-thio-α-D mannopyranoside and cholesteryl(4′-trimethyl 35 ammonio)butanoate.

Additional lipid compounds that may be used include tocopherol and derivatives, and oils and derivatized oils such as stearlyamine.

A variety of cationic lipids such as DOTMA, N-[1-(2,3-dioleoyloxy)propyl-N,N,N-trimethylammonium chloride; DOTAP, 1,2-dioleoyloxy-3-(trimethylammonio) propane; and DOTB, 1,2-dioleoyl-3-(4′-trimethyl-ammonio) butanoyl-sn glycerol may be used.

A variety of other surfactants may be used including ethoxylated sorbitan esters, sorbitan esters, fatty acid salts, sugar esters, pluronics, tetronics, ethylene oxides, butylene oxides, propylene oxides, anionic surfactants, cationic surfactants, mono and diacyl glycerols, mono and diacyl ethylene glycols, mono and diacyl sorbitols, mono and diacyl glycerol succinates, alkyl acyl phosphatides, fatty alcohols, fatty amines and their salts, fatty ethers, fatty esters, fatty amides, fatty carbonates, cholesterol esters, cholesterol amides and cholesterol ethers.

Examples of anionic or cationic surfactants include aluminum monostearate, ammonium lauryl sulfate, calcium stearate, dioctyl calcium sulfosuccinate, dioctyl potassium sulfosuccinate, dioctyl sodium sulfosuccinate, emulsifying wax, magnesium lauryl sulfate, potassium oleate, sodium caster oil, sodium cetostearyl sulfate, sodium lauryl ether sulfate, sodium lauryl sulfate, sodium lauryl sulfoacetate, sodium oleate, sodium stearate, sodium stearyl fumarate, sodium tetradecyl sulfate, zinc oleate, zinc stearate, benzalconium chloride, cetrimide, cetrimide bromide, and cetylpyridinium chloride.

Pharmaceutical Agent

A wide variety of pharmaceutical agents can be loaded within the porous microparticles of the sustained release formulations described herein. The “pharmaceutical agent” is a therapeutic, diagnostic, or prophylactic agent. It may be referred to herein generally as a “drug” or “active agent.” The pharmaceutical agent can be, for example, a protein, peptide, sugar, oligosaccharide, nucleic acid molecule, or other synthetic or natural agent. The pharmaceutical agent may be present in an amorphous state, a crystalline state, or a mixture thereof.

Representative examples of suitable pharmaceutical agents include the following categories and examples of pharmaceutical agents and alternative forms of these pharmaceutical agents such as alternative salt forms, free acid forms, free base forms, and hydrates:

analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, fentanyl, and meprobamate);

antiasthmatics (e.g., xanthines such as theophylline, aminophylline, dyphylline, metaproterenol sulfate, and aminophylline; mast cell stabilizers such as cromolyn sodium and nedocromil sodium; anticholinergic agents such as ipratropium bromide; inhalant corticosteroids such as budesonide, beclomethasone dipropionate, flunisolide, triamcinolone acetonide, mometasone, and fluticasone propionate; leukotriene modifiers such as zafirlukast and zileuton; corticosteroids such as methyl prednisolone, prednisolone, prednisone, ketotifen, and traxanox);

antibiotics (e.g., neomycin, streptomycin, chloramphenicol, cephalosporin, ampicillin, penicillin, tetracycline, and ciprofloxacin);

antidepressants (e.g., nefopam, oxypertine, doxepin, amoxapine, trazodone, amitriptyline, maprotiline, phenelzine, desipramine, nortriptyline, tranylcypromine, fluoxetine, imipramine, imipramine pamoate, isocarboxazid, trimipramine, and protriptyline);

antidiabetics (e.g., biguanides and sulfonylurea derivatives);

antifungal agents (e.g., griseofulvin, ketoconazole, itraconizole, amphotericin B, nystatin, voriconazole, and candicidin);

antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine);

anti-inflammatories (e.g., (non-steroidal) indomethacin, ketoprofen, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone);

antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof vinblastine, vincristine, tamoxifen, and piposulfan);

antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene);

immunosuppressive agents (e.g., cyclosporine, azathioprine, mizoribine, and FK506 (tacrolimus));

antimigraine agents (e.g., ergotamine, propanolol, isometheptene mucate, and dichloralphenazone);

sedatives/hypnotics (e.g., barbiturates such as pentobarbital, pentobarbital, and secobarbital; and benzodiazapines such as flurazepam hydrochloride, triazolam, and midazolam);

antianginal agents (e.g., beta-adrenergic blockers; calcium channel blockers such as nifedipine, and diltiazem; and nitrates such as nitroglycerin, isosorbide dinitrate, pentaerythritol tetranitrate, and erythrityl tetranitrate);

antipsychotic agents (e.g., haloperidol, haloperidol decanoate, loxapine succinate, loxapine hydrochloride, thioridazine, thioridazine hydrochloride, thiothixene, thioxthixene hydrochloride, pimozide, risperidone, quetiapine fumarate, olanzapine, fluphenazine, fluphenazine decanoate, fluphenazine enanthate, trifluoperazine, chlorpromazine, perphenazine, lithium citrate, clozapine, ziprasidone hydrochloride, ziprasidone mesylate, molidone hydrochloride and prochlorperazine);

antimanic agents (e.g., lithium carbonate);

antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine);

antiarthritic agents (e.g., phenylbutazone, sulindac, penicillamine, salsalate, piroxicam, azathioprine, indomethacin, meclofenamate, gold sodium thiomalate, ketoprofen, auranofin, aurothioglucose, and tolmetin sodium);

antigout agents (e.g., colchicine, and allopurinol);

anticoagulants (e.g., heparin, heparin sodium, and warfarin sodium);

thrombolytic agents (e.g., urokinase, streptokinase, and alteplase);

atifibrinolytic agents (e.g., aminocaproic acid);

hemorheologic agents (e.g., pentoxifylline);

antiplatelet agents (e.g., aspirin);

anticonvulsants (e.g., valproic acid, divalproex sodium, phenyloin, phenyloin sodium, clonazepam, primidone, phenobarbitol, carbamazepine, amobarbital sodium, methsuximide, metharbital, mephobarbital, mephenyloin, phensuximide, paramethadione, ethotoin, phenacemide, secobarbitol sodium, clorazepate dipotassium, and trimethadione);

antiparkinson agents (e.g., ethosuximide);

antihistamines/antiprurities (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, and methdilazine);

agents useful for calcium regulation (e.g., calcitonin, and parathyroid hormone);

antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate);

antiviral agents (e.g., interferon alpha, beta or gamma, zidovudine, amantadine hydrochloride, ribavirin, and acyclovir);

antimicrobials (e.g., cephalosporins such as cefazolin sodium, cephradine, cefaclor, cephapirin sodium, ceftizoxime sodium, cefoperazone sodium, cefotetan disodium, cefuroxime azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin);

anti-infectives (e.g., GM-CSF);

bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine, and epinephrine bitartrate, salbutamol, formoterol, salmeterol, xinafoate, and pirbuterol);

steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium);

hypoglycemic agents (e.g., human insulin, purified beef insulin, purified pork insulin, glyburide, chlorpropamide, glipizide, tolbutamide, and tolazamide);

hypolipidemic agents (e.g., clofibrate, dextrothyroxine sodium, probucol, pravastitin, atorvastatin, lovastatin, and niacin);

proteins (e.g., DNase, alginase, superoxide dismutase, interferons, growth hormone, follicle stimulating hormone, interleukins, thrombopoietin, antibodies, and lipase);

nucleic acids (e.g., sense or anti-sense nucleic acids encoding any therapeutically useful protein, including any of the proteins described herein);

agents useful for erythropoiesis stimulation (e.g., erythropoietin);

antiulcer/antireflux agents (e.g., famotidine, cimetidine, and ranitidine hydrochloride);

antinauseants/antiemetics (e.g., meclizine hydrochloride, nabilone, prochlorperazine, dimenhydrinate, promethazine hydrochloride, thiethylperazine, ondansetron hydrochloride, palonsetron hydrochloride, and scopolamine);

oil-soluble vitamins (e.g., vitamins A, D, E, K, and the like);

as well as other pharmaceutical agents such as mitoxotrane, halonitrosoureas, anthrocyclines, and ellipticine. A description of these and other classes of useful pharmaceutical agents and a listing of species within each class can be found in Martindale, The Extra Pharmacopoeia, 30th Ed. (The Pharmaceutical Press, London 1993).

In one embodiment, the pharmaceutical agent comprises a steroid, such as testosterone, progesterone, and estradiol.

In another embodiment, the pharmaceutical agent comprises an antipsychotic (such as haloperidol, haloperidol decanoate, loxapine succinate, loxapine hydrochloride, thioridazine, thioridazine hydrochloride, thiothixene, thioxthixene hydrochloride, pimozide, risperidone, quetiapine fumarate, olanzapine, fluphenazine, fluphenazine decanoate, fluphenazine enanthate, trifluoperazine, chlorpromazine, perphenazine, lithium citrate, clozapine, ziprasidone hydrochloride, ziprasidone mesylate, molidone hydrochloride and prochlorperazine), an analgesic (such as morphine and oxydocone), an antiemetic (such as prochlorperazine, ondansetron hydrochloride, and palonsetron hydrochloride), an antibiotic (such as cefprozil, ciprofloxacin, and amoxicillin), an antifungal (such as voriconazole and itraconazole), an antineoplastic (such as paclitaxel and docetaxel), or a peptide or protein (such as insulin, calcitonin, leuprolide, granulocyte colony-stimulating factor, parathyroid hormone-related peptide, growth hormone, interferons, erythropoietin, follicle stimulating hormone, interleukins, thrombopoietin, antibodies and somatostatin).

The content of pharmaceutical agent in the microparticles generally is between about 1 and about 70 wt %. In typical embodiments, the pharmaceutical agent is present in an amount between about 5 and 50 wt %.

In one embodiment, the sustained release formulations comprise two or more different pharmaceutical agents. In one embodiment, two or more pharmaceutical agents are combined into and delivered from one microparticle. In another embodiment, the formulation comprises a mixture of two or more different microparticles each containing a different pharmaceutical agent or pharmaceutical agents. In one embodiment, the formulation includes at least one pharmaceutical agent for sustained release and at least one other pharmaceutical agent for immediate release.

In yet another embodiment, the sustained release formulations comprise a mixture of different microparticles each containing a single pharmaceutical agent, but having different porosities, so that the some particles of the mixture have a first release profile (e.g., a majority of the first pharmaceutical agent is released between 2 and 24 hours) and other particles have a second pharmaceutical agent release profile (e.g., a majority of the second pharmaceutical agent is released after 24 hours).

### Materials to Inhibit Uptake by the RES

Uptake and removal of the microparticles by macrophages can be slowed or minimized through increasing the geometric particle size (e.g., >3 μm slows phagocytosis) the selection of the polymer and/or incorporation or coupling of molecules that minimize adhesion or uptake or by incorporating the poly(alkylene glycol) into the matrix such that at least one glycol unit is surface exposed. For example, tissue adhesion by the microparticle can be minimized by covalently binding poly(alkylene glycol) moieties to the surface of the microparticle. The surface poly(alkylene glycol) moieties have a high affinity for water that reduces protein adsorption onto the surface of the particle. The recognition and uptake of the microparticle by the reticulo-endothelial system (RES) is therefore reduced.

In one method, the terminal hydroxyl group of the poly(alkylene glycol) is covalently attached to biologically active molecules, or molecules affecting the charge, lipophilicity or hydrophilicity of the particle, onto the surface of the microparticle. Methods available in the art can be used to attach any of a wide range of ligands to the microparticles to enhance the delivery properties, the stability or other properties of the microparticles in vivo.

Pharmaceutically Acceptable Vehicle for Injection

For administration by injection, the porous microparticles typically are combined with (e.g., suspended in) one or more pharmaceutically acceptable vehicles for injection. The pharmaceutically acceptable vehicle can be any aqueous or non-aqueous vehicle known in the art. Examples of aqueous vehicles include physiological saline solutions, solutions of sugars such as dextrose or mannitol, and pharmaceutically acceptable buffered solutions, and examples of non-aqueous vehicles include fixed vegetable oils, glycerin, polyethylene glycols, alcohols, and ethyl oleate. The vehicle may further include antibacterial preservatives, antioxidants, tonicity agents, buffers, stabilizers, or other components.

Formulation Additives for Topical Administration

For topical administration, the porous microparticles are combined with one or more additives selected from among the various pharmaceutically acceptable topical dosage form additives available to those skilled in the art. These additives include ointment, gel, or paste base materials, binders, stabilizers, preservatives, flavorings, bioadhesive polymers or other bioadhesive materials, and pigments. For topical administration, the porous microparticles may be a component of a transdermal delivery system such as a patch.

Formulation Additives for Oral Administration

For oral administration, the porous microparticles are combined with one or more additives selected from among the various pharmaceutically acceptable oral dosage form additives available to those skilled in the art. These additives include binders, taste modifying components, food colorings, and viscosity modifying agents. The drug formulation may be in the form of a suspension, capsule, tablet, paste, gel, or solid or semi-solid form. For example, the microparticles can be suspended in an aqueous solution containing sweeteners and/or flavoring agents, which are well known in the art. Some dosage forms may be enterically coated to delay initiation of release of the pharmaceutical agent.

Making the Porous Microparticles and Sustained Release Formulations

In typical embodiments, the porous microparticles are made by a method that includes the following steps: (1) dissolving the matrix material in a volatile solvent to form a matrix material solution; (2) adding the pharmaceutical agent to the solution of matrix material; (3) optionally combining at least one pore forming agent with the pharmaceutical agent in the matrix material solution and emulsifying to form an emulsion, suspension, or second solution; and (4) removing the volatile solvent, and the pore forming agent if present, from the emulsion, suspension, or second solution to yield porous microparticles which comprise the pharmaceutical agent and the matrix material. The method produces microparticles that release a therapeutically or prophylactically effective amount of the pharmaceutical agent from the microparticles in the body for at least 2 hours. Techniques that can be used to make the porous microparticles include melt extrusion, spray drying, fluid bed drying, solvent extraction, hot melt encapsulation, and solvent evaporation, as discussed below. In the most preferred embodiment, microparticles are produced by spray drying. The pharmaceutical agent can be incorporated into the matrix as solid particles, liquid droplets, or by dissolving the pharmaceutical agent in the matrix material solvent. If the pharmaceutical agent is a solid, the pharmaceutical agent may be encapsulated as solid particles which are added to the matrix material solution or may be dissolved in an aqueous solution which then is emulsified with the matrix material solution prior to encapsulation, or the solid pharmaceutical agent may be cosolubilized together with the matrix material in the matrix material solvent.

In one embodiment, the method further comprises combining one or more surfactants, with the pharmaceutical agent in a matrix material solution. In one embodiment of the methods for making sustained release formulations, the process further includes blending the porous microparticles with a pharmaceutically acceptable bulking agent.

In one example, the matrix material comprises a biocompatible synthetic polymer, and the volatile solvent comprises an organic solvent. In another example, the pore forming agent is in the form of an aqueous solution when combined with the pharmaceutical agent/matrix solution.

In one embodiment, the step of removing the volatile solvent and pore forming agent from the emulsion, suspension, or second solution is conducted using a process selected from spray drying, evaporation, fluid bed drying, lyophilization, vacuum drying, or a combination thereof.

Solvent Evaporation

In this method, the matrix material and pharmaceutical agent are dissolved in a volatile organic solvent such as methylene chloride. A pore forming agent as a solid or as a liquid may be added to the solution. The active agent can be added as either a solid or in solution to the polymer solution. The mixture is sonicated or homogenized and the resulting dispersion or emulsion is added to an aqueous solution that may contain a surface active agent such as TWEEN™ 20, TWEEN™ 80, PEG or poly(vinyl alcohol) and homogenized to form an emulsion. The resulting emulsion is stirred until most of the organic solvent evaporates, leaving microparticles. Microparticles with different geometric sizes and morphologies can be obtained by this method by controlling the emulsion droplet size. Solvent evaporation is described by Mathiowitz, et al., J. Scanning Microscopy, 4:329 (1990); Beck, et al., Fertil. Steril., 31:545 (1979); and Benita, et al., J. Pharm. Sci., 73:1721 (1984).

Particularly hydrolytically unstable polymers, such as polyanhydrides, may degrade during the fabrication process due to the presence of water. For these polymers, the following two methods, which are performed in completely organic solvents, are more useful.

Hot Melt Microencapsulation

In this method, the matrix material and the pharmaceutical agent are first melted and then mixed with the solid or liquid active agent. A pore forming agent as a solid or in solution may be added to the solution. The mixture is suspended in a non-miscible solvent (like silicon oil), and, while stirring continuously, heated to 5° C. above the melting point of the polymer. Once the emulsion is stabilized, it is cooled until the polymer particles solidify. The resulting microparticles are washed by decantation with a polymer non-solvent such as petroleum ether to give a free-flowing powder. Hot-melt microencapsulation is described by Mathiowitz, et al., Reactive Polymers, 6:275 (1987).

Solvent Removal

This technique was primarily designed for hydrolytically unstable materials. In this method, the solid or liquid pharmaceutical agent is dispersed or dissolved in a solution of the selected matrix material and pharmaceutical agent in a volatile organic solvent like methylene chloride. This mixture is suspended by stirring in an organic oil (such as silicon oil) to form an emulsion. The external morphology of particles produced with this technique is highly dependent on the type of polymer used.

Spray Drying of Microparticles

Microparticles can be produced by spray drying by a method that includes the following steps: (1) dissolving the matrix material, and optionally a surfactant, in a volatile solvent to form a matrix material solution; (2) adding a pharmaceutical agent to the solution of matrix material; (3) optionally combining at least one pore forming agent with the pharmaceutical agent in the matrix material solution; (4) forming an emulsion, suspension or second solution from the pharmaceutical agent, the matrix material solution, and the optional pore forming agent; and (5) spray drying the emulsion, suspension or solution and removing the volatile solvent and the pore forming agent, if present, to form porous microparticles. As defined herein, the process of “spray drying” an emulsion, suspension or solution containing a matrix material and a pharmaceutical agent refers to a process wherein the emulsion, suspension or solution is atomized to form a fine mist and dried by direct contact with temperature-controlled carrier gases. In a typical embodiment using spray drying apparatus available in the art, the emulsion, suspension or solution is delivered through the inlet port of the spray drier, passed through a tube within the drier and then atomized through the outlet port. The temperature may be varied depending on the gas or matrix material used. The temperature of the inlet and outlet ports can be controlled to produce the desired products.

The geometric size of the particulates formed is a function of the atomizer used to spray the matrix material solution, atomizer pressure, the flow rate, the matrix material used, the matrix material concentration, the type of solvent and the temperature of spraying (both inlet and outlet temperature). Microparticles ranging in geometric diameter between one and ten microns can be obtained.

If the pharmaceutical agent is a solid, the agent may be encapsulated as solid particles which are added to the matrix material solution prior to spraying, or the pharmaceutical agent can be dissolved in a solvent which then is emulsified with the matrix material solution prior to spraying, or the solid may be cosolubilized together with the matrix material in an appropriate solvent prior to spraying.

Reagents for Making the Porous Microparticles

Certain reagents used to make the porous microparticles may include solvents for the matrix material, solvents or vehicles for the pharmaceutical agent, pore forming agents, and various additives to facilitate microparticle formation.

Solvents

A solvent for the matrix material is selected based on its biocompatibility as well as the solubility of the matrix material and where appropriate, interaction with the pharmaceutical agent to be delivered. For example, the ease with which the matrix material is dissolved in the solvent and the lack of detrimental effects of the solvent on the pharmaceutical agent to be delivered are factors to consider in selecting the matrix material solvent. Aqueous solvents can be used to make matrices formed of water-soluble polymers. Organic solvents will typically be used to dissolve hydrophobic and some hydrophilic matrix materials. Combinations of aqueous and organic solvents may be used. Preferred organic solvents are volatile or have a relatively low boiling point or can be removed under vacuum and which are acceptable for administration to humans in trace amounts, such as methylene chloride. Other solvents, such as ethyl acetate, ethanol, methanol, dimethyl formamide (DMF), acetone, acetonitrile, tetrahydrofuran (THF), acetic acid, dimethyl sulfoxide (DMSO) and chloroform, and combinations thereof, also may be utilized. Preferred solvents are those rated as class 3 residual solvents by the Food and Drug Administration, as published in the Federal Register vol. 62, number 85, pp. 24301-09 (May 1997).

In general, the matrix material is dissolved in the solvent to form a matrix material solution having a concentration of between 0.1 and 60% weight to volume (w/v), more preferably between 0.25 and 30%. The matrix material solution is then processed as described below to yield a matrix having pharmaceutical agents incorporated therein.

Surfactants to Facilitate Micro Particle Formation

A variety of surfactants may be added to a solution, suspension, or emulsion containing matrix material to facilitate microparticle formation. The surfactants may be added to any phase of an emulsion as emulsifiers if an emulsion is used during the production of the matrices. Exemplary emulsifiers or surfactants that may be used (e.g., between about 0.1 and 5% by weight relative to weight of the pharmaceutical agent and matrix material) include most physiologically acceptable emulsifiers. Examples include natural and synthetic forms of bile salts or bile acids, both conjugated with amino acids and unconjugated such as taurodeoxycholate, and cholic acid. Phospholipids can be used as mixtures, including natural mixtures such as lecithins. These surfactants may function solely as emulsifiers, and as such form part of and are dispersed throughout the matrix of the particles.

Additives to Facilitate Microparticle Suspension

The composition of the microparticles may comprise an additive in a manner such that the microparticles will have all or part of the additive structure surface exposed, and as such will facilitate suspension of the microparticles in a vehicle for administration. Additives for facilitating suspension may be included during production of the microparticles. Alternatively, the microparticles may be coated with the additive post-production. Exemplary additives include surfactants that may be used (e.g., between about 0.1 and 5% by weight relative to weight of the pharmaceutical agent and matrix material) include phospholipids, salts of fatty acids, and molecules containing PEG units such as polysorbate 80.

Control of Porosity

The porosity of the microparticles can be controlled during the production of the microparticles by adjusting the solids content of the pharmaceutical agent in matrix material solution or adjusting the rate at which the matrix solvent is removed, or combinations thereof. Higher solids concentrations lead to microparticles with less porosity.

Alternatively, pore forming agents as described below can be used to control the porosity of the microparticles during production. Pore forming agents are volatile materials that are used during the process to create porosity in the resultant matrix. The pore forming agent can be a volatilizable solid or volatilizable liquid.

Porosity is created during the production and formation of the microparticles.

Liquid Pore Forming Agent

The liquid pore forming agent must be immiscible with the matrix material solvent and volatilizable under processing conditions compatible with the pharmaceutical agent and matrix material. To effect pore formation, the pore forming agent first is emulsified with the pharmaceutical agent in the matrix material solution. Then, the emulsion is further processed to remove the matrix material solvent and the pore forming agent simultaneously or sequentially using evaporation, vacuum drying, spray drying, fluid bed drying, lyophilization, or a combination of these techniques.

The selection of liquid pore forming agents will depend on the matrix material solvent. Representative liquid pore forming agents include water; dichloromethane; alcohols such as ethanol, methanol, or isopropanol; acetone; ethyl acetate; ethyl formate; dimethylsulfoxide; acetonitrile; toluene; xylene; dimethylforamide; ethers such as THF, diethyl ether, or dioxane; triethylatnine; foramide; acetic acid; methyl ethyl ketone; pyridine; hexane; pentane; furan; water; liquid perfluorocarbons, and cyclohexane.

The liquid pore forming agent is used in an amount that is between 1 and 50% (v/v), preferably between 5 and 25% (v/v), of the pharmaceutical agent solvent emulsion.

Solid Pore Forming Agent

The solid pore forming agent must be volatilizable under processing conditions which do not harm the pharmaceutical agent or matrix material. The solid pore forming agent can be (i) dissolved in the matrix material solution which contains the pharmaceutical agent, (ii) dissolved in a solvent which is not miscible with the matrix material solvent to form a solution which is then emulsified with the matrix material solution which contains the pharmaceutical agent, or (iii) added as solid particulates to the matrix material solution which contains the pharmaceutical agent. The solution, emulsion, or suspension of the pore forming agent in the pharmaceutical agent/matrix material solution then is further processed to remove the matrix material solvent, the pore forming agent, and, if appropriate, the solvent for the pore forming agent simultaneously or sequentially using evaporation, spray drying, fluid bed drying, lyophilization, vacuum drying, or a combination of these techniques. After the matrix material is precipitated, the hardened microparticles can be frozen and lyophilized to remove any pore forming agents not removed during the microencapsulation process.

In a preferred embodiment, the solid pore forming agent is a volatile salt, such as salts of volatile bases combined with volatile acids. Volatile salts are materials that can transform from a solid or liquid to a gaseous state using added heat and/or vacuum. Examples of volatile bases include ammonia, methylamine, ethylamine, dimethylamine, diethylamine, methylethylamine, trimethylamine, triethylamine, and pyridine. Examples of volatile acids include carbonic acid, hydrochloric acid, hydrobromic acid, hydroiodic acid, formic acid, acetic acid, propionic acid, butyric acid, and benzoic acid. Preferred volatile salts include ammonium bicarbonate, ammonium acetate, ammonium chloride, ammonium benzoate and mixtures thereof. Other examples of solid pore forming agents include iodine, phenol, benzoic acid (as acid not as salt), camphor, and naphthalene.

The solid pore forming agent is used in an amount between 5 and 1000% (w/w), preferably between 10 and 600% (w/w), and more preferably between 10 and 100% (w/w), of the pharmaceutical agent and the matrix material.

Methods of Administering the Porous Microparticles

The sustained release formulations described herein can be designed for administration to patients by injection, by oral administration, or by topical administration. As used herein, “patient” refers to animals, including mammals, preferably humans.

Administration by Injection

The sustained release formulations comprising porous microparticles described herein can be administered to a patient by injection in a pharmaceutically acceptable vehicle, for local, regional, or systemic delivery of the pharmaceutical agent. An injection is typically carried out using conventional syringes and needles, catheters, and the like. In other embodiments, the formulations can be injected by more complex delivery systems, such as needleless injectors. The pharmaceutical formulation may be injected into almost any organ or area of the body, including by intravenous, intramuscular, intracutaneous, subcutaneous, intra-articular, intrasynovial, intraosseous intraspinal, intrathecal, intra-arterial, or intracardiac administration. In still other embodiments, the formulation is suitable for intracranial, intralesional, or intratumoral administration.

In one embodiment, the porous microparticles are in the form of powder, which can be stably stored, reconstituted with a vehicle immediately before use, and administered by injection. In such a case, the formulation and vehicle may be provided or packaged in a kit form.

Topical Administration

The sustained release formulations comprising porous microparticles described herein can be administered to a patient by topical application in a suitable semi-solid dosage form, for local, regional, or systemic delivery of the pharmaceutical agent. The term “topical” or “topically” is used herein refers to an area on any part of the body, including the skin or a mucosal membrane surface. For example, the microparticle formulation may be in the form of a paste or ointment for application to an area of the patient's skin for sustained release and local delivery of a corticosteroid or an analgesic such as fentanyl to the patient. As another example, the microparticle formulation may be in the form of a gel for application to vaginal mucosal tissues for sustained release and local delivery of an antifungal agent. As another example, the microparticle formulation may be a component of a transdermal patch for sustained release and systemic delivery of a steroid such as estradiol or an analgesic such as morphine.

Oral Administration

The sustained release formulations comprising porous microparticles described herein can be administered to a patient by oral application in a suitable oral dosage form, for local or systemic delivery of the pharmaceutical agent. The microparticles can be loaded into gelatin capsules, possibly formed into tablets or wafers, or other solid delivery forms, or the microparticles can be suspended in a liquid vehicle to form a suspension, using materials and methods well know in the art. Administration simply requires that the patient ingest the oral formulation.

The methods and compositions described above will be further understood with reference to the following non-limiting examples.

### EXAMPLES

In the examples below, where porosity of microparticles was determined, the following procedure was used: TAP Density (Transaxial Pressure Density as a measure of tap density) for the microparticles was determined using a Micromeritics GeoPyc Model 1360. Envelope density for the microparticles was estimated from the TAP density (EQ.5). Absolute density was determined via helium pycnometry using a Micromeritics AccuPyc Model 1330. The absolute densities of the polymer, pharmaceutical agent, and phospholipid were determined, and a weighted average value was used for the absolute density of the microparticles. The porosity was calculated based on EQ.6 above. Where percent porosity is reported, the value of porosity (based on EQ.6) was multiplied by 100%.

In the examples below, the in vitro pharmaceutical agent release rate was determined using the following procedure. Microparticles were suspended in PBS-SDS (Phosphate Buffered Saline—0.05% Sodium Dodecyl Sulfate) such that the nominal pharmaceutical agent concentration in the suspension was 1 mg/mL. A sample of the suspension was then added to a large volume of PBS-SDS at 37° C., such that theoretical pharmaceutical agent concentration at 100% release was 0.75 μg/mL. The resulting diluted suspension was maintained at 37° C. in an incubator on a rocker. To determine the release rate of pharmaceutical agent from the microparticles, samples of the release media were taken over time, the microparticles separated from the solution, and the solution pharmaceutical agent concentration was monitored via HPLC with detection at 254 nm for budesonide or 238 nm for fluticasone propionate. The column was a J'Sphere ODS-H80 (250×4.6 mm, 4 μm). The mobile phase was an isocratic system consisting of Ethanol-Water (64:36), running at a flow rate of 0.8 mL/min.

In the examples below, where geometric particle size is described, the volume average size was measured using a Coulter Multisizer II with a 50 μm aperture.

Powders were dispersed in an aqueous vehicle containing Pluronic F127 and mannitol using vortexing and sonication. The resulting suspensions were then diluted into electrolyte for analysis.

### Example 1

### Effect of Microparticle Porosity on Budesonide Release

Microspheres containing budesonide were prepared, using materials obtained as follows: budesonide was from FarmaBios S.R.L. (Pavia, Italy); phospholipid (DPPC) was from Avanti Polar Lipids Inc. (Alabaster, ΔL); polymer (PLGA) was from BI Chemicals (Petersburg, Va.); ammonium bicarbonate was from Spectrum Chemicals (Gardena, Calif.); and methylene chloride was from EM Science (Gibbstown, N.J.).

Six different lots of budesonide containing microspheres (B1 through B6) were prepared as follows. For each microsphere lot (B1-B4 and B6) 8.0 g of PLGA, 0.72 g of DPPC, and 2.2 g of budesonide were dissolved into 364 mL of methylene chloride at 20° C. For lot B5, 36.0 g of PLGA, 2.16 g of DPPC, and 9.9 g of budesonide were dissolved into 1764 mL of methylene chloride at 20° C. Lot B1 was prepared without a pore forming agent, and the process conditions and solids content of the solution to the spray dryer were used to create the porosity of the microspheres. Lots B2-B6 were prepared using the pore forming agent, ammonium bicarbonate to create microspheres having porosities greater than lot B1. For lots B2-B6, a stock solution of the pore forming agent was prepared by dissolving 4.0 g of ammonium bicarbonate into 36 mL of RO/DI water at 20° C. For each lot, a different ratio of the ammonium bicarbonate stock solution was combined with the pharmaceutical agent/polymer solution (volume pore forming agent: pharmaceutical agent/polymer solution: B2: 1:49, B3: 1:24, B4: 1:10, B5: 1:49, B6: 1:19) described above and emulsified using a rotor-stator homogenizer. The resulting emulsion was spray dried on a benchtop spray dryer using an air-atomizing nozzle and nitrogen as the drying gas. Spray drying conditions were as follows: 20 mL/min emulsion flow rate, 60 kg/hr drying gas rate and 21° C. outlet temperature. The product collection container was detached from the spray dryer and attached to a vacuum pump, where it was dried for at least 18 hours.

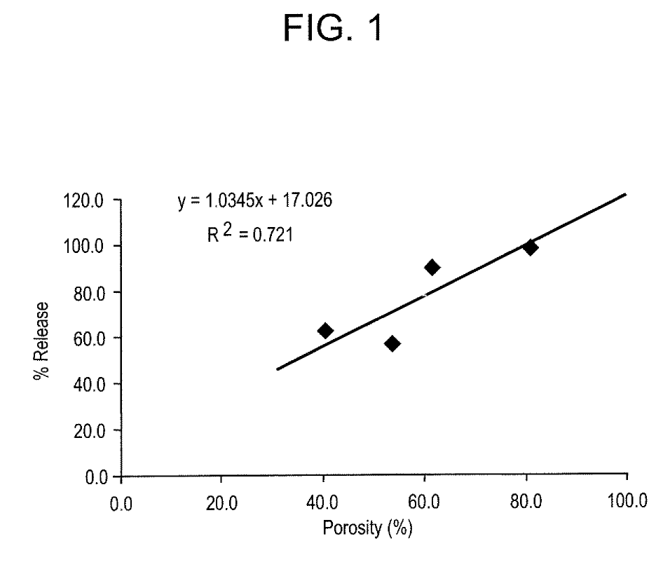

FIG. 1 is a graph of percent of budesonide released in vitro after 5.5 hours versus porosity. Table 1 shows the geometric size, density and porosity data for the lots shown in FIG. 1.

TABLE 1
Geometric Size, Tap Density and Porosity
Of the Budesonide-Containing Microspheres

| | Geometric Size | Tap density | |
|---|---|---|---|
| Lot # | (μm) | (g/mL) | Porosity × 100% |
| B4 | 2.3 | 0.22 | 81 |
| B3 | 2.1 | 0.44 | 61 |
| B2 | 2.5 | 0.53 | 53 |
| B1 | 1.7 | 0.68 | 40 |

Table 2 further illustrates the effect of porosity on the percent budesonide released after 24 hours.

TABLE 2
Effect of Porosity on Budesonide Release After 24 Hours

| Lot # | Porosity × 100% | % Budesonide release after 24 hours |
|---|---|---|
| B6 | 57.8 | 86.5 |
| B5 | 46.1 | 58.9 |

The in vitro budesonide release data demonstrate how the control of porosity can be used to to adjust the amount of pharmaceutical agent released after a certain period of time, and how porosity can be used to ensure that significant release of the pharmaceutical agent occurs beyond the initial release and that the release of the pharmaceutical agent is occurring over at least 24 hours.

### Example 2

### Effect of Microparticle Porosity on Fluticasone Propionate Release

Microspheres containing fluticasone propionate were prepared, using materials obtained as follows: fluticasone propionate was from Cipla Ltd. (Mumbai, India); phospholipid (DPPC) was from Chemi S.p.A. (Milan, Italy); polymer (PLGA) was from BI Chemicals (Petersburg, Va.); ammonium bicarbonate was from Spectrum Chemicals (Gardena, Calif.): and methylene chloride was from EM Science (Gibbstown, N.J.).

Six different lots of fluticasone proprionate containing microspheres (F1 through F6) were prepared as follows. For each microsphere lot, 3.0 g of PLGA, 0.18 g of DPPC, and 0.825 g of fluticasone propionate were dissolved into 136.4 mL of methylene chloride at 20° C. Lot F1 was prepared without a pore forming agent, and the process conditions and solids content of the solution to the spray dryer were used to create the porosity of the microspheres. Lots F2-F6 were prepared using the pore forming agent ammonium bicarbonate to create microspheres having porosities greater than lot F1. A stock solution of the pore forming agent was prepared by dissolving 2.22 g of ammonium bicarbonate into 20 g of RO/DI water at 20° C. For each lot, a different ratio of ammonium bicarbonate stock solution was combined with the pharmaceutical agent/polymer solution (volume ammonium bicarbonate solution: volume pharmaceutical agent/polymer solution: F2: 1:74, F3: 1:49, F4: 1:24, F5: 1:14, F6: 1:10) and the mixture was then emulsified using a rotor-stator homogenizer. The resulting emulsion was spray dried on a benchtop spray dryer using an air-atomizing nozzle and nitrogen as the drying gas. Spray drying conditions were as follows: 20 mL/min emulsion flow rate, 60 kg/hr drying gas rate, and 21° C. outlet temperature. The product collection container was detached from the spray dryer and attached to a vacuum pump, where it was dried for at least 18 hours.

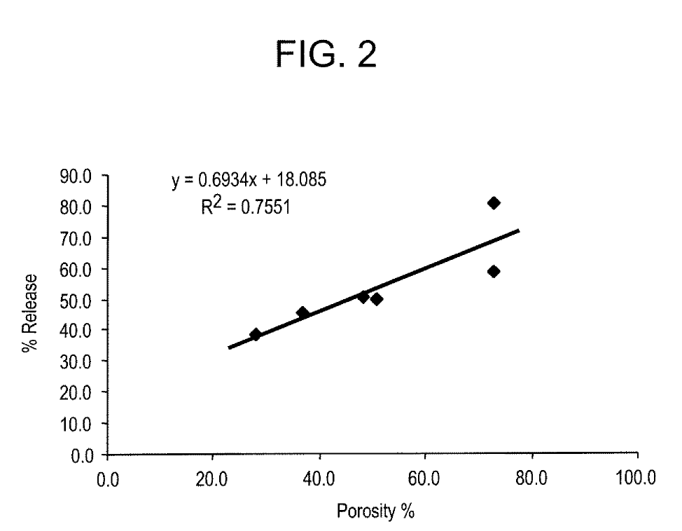

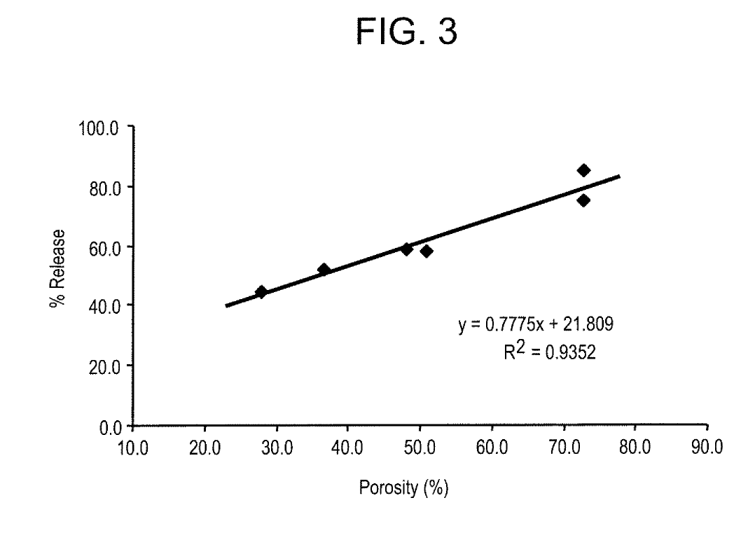

FIGS. 2 and 3 are graphs of percent of fluticasone released in vitro after 5.5 hours and 24 hours, respectively, versus porosity. Table 3 shows the geometric size, density, and porosity data for the material whose release is shown in FIGS. 2 and 3.

TABLE 3
Geometric Size, Tap Density, and Porosity
Of the Fluticasone Propionate-Containing Microspheres

| Lot # | Geometric Size (μm) | Tap density (g/mL) | Porosity × 100% |
|---|---|---|---|
| F6 | 3.8 | 0.31 | 73 |
| F5 | 3.5 | 0.31 | 73 |
| F4 | 3.4 | 0.56 | 51 |
| F3 | 2.7 | 0.59 | 48 |
| F2 | 3.1 | 0.72 | 37 |
| F1 | 3.1 | 0.82 | 28 |

The in vitro fluticasone propionate release data demonstrate how porosity can be used to adjust the amount of pharmaceutical agent released after a certain period of time and can be used to ensure that significant release of the pharmaceutical agent.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

We claim:

1. A method for making an injectable, sustained release pharmaceutical formulation comprising:
dissolving a hydrophobic matrix material in a volatile solvent to form a first solution;
adding a pharmaceutical agent to the first solution to form an emulsion, suspension, or second solution; and
removing the volatile solvent from the emulsion, suspension, or second solution to yield porous microparticles which comprise the pharmaceutical agent dispersed, entrapped or encapsulated within the structure of the hydrophobic matrix material,
wherein the size, porosity, matrix material, and pharmaceutical agent of the microparticles together provide release of a therapeutically or prophylactically effective amount of the pharmaceutical agent from the microparticles for at least 24 hours following injection of a formulation comprising said microparticles.

2. The method of claim 1, wherein the matrix material comprises a synthetic polymer.

3. The method of claim 2, wherein the synthetic polymer is selected from the group consisting of polyhydroxy acids, polyanhydrides, polyorthoesters, polyamides, polycarbonates, polyalkylenes, polyalkylene terepthalates, polyvinyl ethers, polyvinyl esters, polyvinyl halides, polysiloxanes, polystyrene, polyurethanes, polybutyric acid, polyvaleric acid, poly(lactide-co-caprolactone), and copolymers, derivatives, and blends thereof.

4. The method of claim 1, further comprising the step of combining one or more surfactants with the first solution.

5. The method of claim 4, wherein the surfactant comprises a phospholipid.

6. The method of claim 1, wherein the porous microparticles have a volume average diameter between about 1 μm and 150 μm.

7. The method of claim 1, wherein the porous microparticles have an average porosity between about 25 and about 75% by volume.

8. The method of claim 1, wherein the pharmaceutical agent comprises a peptide, a protein, or an oligonucleotide.

9. The method of claim 1, wherein the pharmaceutical agent comprises a steroid, antipsychotic agent, antineoplastic agent, or antiemetic agent.

10. The method of claim 1, further comprising the step of combining the microparticles with a pharmaceutically acceptable vehicle for injection.

11. The method of claim 1, further comprising blending additional microparticles with the porous microparticles.

12. The method of claim 11, wherein the additional microparticles comprise a second pharmaceutical agent.

13. The method of claim 1, wherein the porous microparticles release a majority of the pharmaceutical agent by 14 days, 28 days, or 6 months, following injection.

14. The method of claim 1, wherein the porous microparticles release a therapeutically or prophylactically effective amount of the pharmaceutical agent for at least 7 days.

15. The method of claim 1, wherein an iterative process is used to provide that the porous microparticles have a pre-selected duration of release of the pharmaceutical agent, the process comprising:
(a) selecting the matrix material, pharmaceutical agent content, and a geometric size of the microparticles to determine the time and amount of initial pharmaceutical agent release,
(b) varying the porosity of the microparticles to adjust the amount of initial pharmaceutical agent release and to provide release of the pharmaceutical agent for a select duration beyond the initial release; and
(c) selecting the geometric particle size and the porosity of the microparticles to avoid or delay any physiological clearance mechanisms that otherwise would remove the microparticles from a selected injection site prior to the microparticles releasing substantially all of the pharmaceutical agent contained therein.

16. A method for making an injectable, sustained release pharmaceutical formulation comprising:
dissolving a matrix material in a volatile solvent to form a first solution;
combining a pharmaceutical agent and at least one pore forming agent with the first solution to form an emulsion, suspension, or second solution; and
removing the volatile solvent and the pore forming agent from the emulsion, suspension, or second solution to yield porous microparticles which comprise the pharmaceutical agent dispersed, entrapped or encapsulated within the structure of the matrix material,
wherein the size, porosity, matrix material, and pharmaceutical agent of the microparticles together provide release of a therapeutically or prophylactically effective amount of the pharmaceutical agent from the microparticles for at least 24 hours following injection of a formulation comprising said microparticles.

17. The method of claim 16, wherein the pore forming agent is in the form of an aqueous solution when combined with the solution comprising matrix material.

18. The method of claim 16, wherein the pore forming agent is a volatile salt.

19. The method of claim 16, the step of removing the volatile solvent and pore forming agent from the emulsion, suspension, or second solution is conducted using a process selected from spray drying, evaporation, fluid bed drying, lyophilization, vacuum drying, or a combination thereof.

20. The method of claim 16, further comprising the step of combining the microparticles with a pharmaceutically acceptable vehicle for injection.

21. A method of delivering a pharmaceutical agent to a patient comprising:
administering to the patient by injection a sustained release pharmaceutical formulation which comprises porous microparticles which comprise a pharmaceutical agent and a hydrophobic matrix material, wherein upon injection of the formulation a therapeutically or prophylactically effective amount of the pharmaceutical agent is released from the microparticles into the patient for at least 24 hours.

22. The method of claim 21, wherein a majority of the pharmaceutical agent is release no earlier than about 24 hours and no later than about 28 days following injection.

23. The method of claim 21, wherein the formulation provides local or plasma concentrations which do not fluctuate by more than a factor of four over the period of sustained release.

24. The method of claim 21, wherein a majority of the pharmaceutical agent is released from the microparticles by 14 days, 28 days, or 6 months following injection.

25. The method of claim 21, wherein a therapeutically or prophylactically effective amount of the pharmaceutical agent is released from the microparticles for at least 7 days following injection.

26. The method of claim 21, wherein a therapeutically or prophylactically effective amount of the pharmaceutical agent is released from the microparticles for at least 14 days following injection.

27. The method of claim 21, wherein a therapeutically or prophylactically effective amount of the pharmaceutical agent is released from the microparticles for at least 28 days following injection.

28. The method of claim 21, wherein the injection is selected from the group consisting of intravenous, intraarterial, intracardiac, intrathecal, intraosseous, intraarticular, intrasynovial, intracutaneous, subcutaneous, intramuscular, and intradermal.

29. The method of claim 21, wherein the injection is intracranial, intralesional, or intratumoral.
